(19) United States
(12) Patent Application Publication
(10) Pub. No.: US 2015/0031118 A1
(43) Pub. Date: Jan. 29, 2015

(54) Acoustic Mass Sensor

(71) Applicant: Avago Technologies General IP (Singapore) Pte. Ltd., Singapore (SG)

(72) Inventor: Mark A. UNKRICH, Emerald Hills, CA (US)

(73) Assignee: Avago Technologies General IP (Singapore) Pte. Ltd., Singapore (SG)

(21) Appl. No.: 13/949,601

(22) Filed: Jul. 24, 2013

(51) Int. Cl. G01N 33/543; G01G 9/00; G01N 29/24; G01G 19/00

(52) U.S. Cl. CPC G01N 33/54373; G01G 19/00; G01G 9/00; G01N 29/2437
USPC 435/287.9; 422/69; 422/82.01

(57) ABSTRACT

A mass sensor has a common RF input, a set of mass sensor cells, and a common hold input. Each of the mass sensor cells has a local input node coupled to the common RF input, a local output node, a mass-dependent RF filter that includes an electroacoustic resonator having receptors immobilized on a surface thereof, an RF detector, and a hold circuit having a local hold input. The RF filter, the RF detector and the hold circuit are connected in series between the local input node and the local output node. The common hold input is coupled to the local hold inputs of the hold circuits of at least a subset of the mass sensor cells.

### BACKGROUND

Mass detection using acoustic sensors is well described in the literature. The sensor element utilizes a resonator made with a piezoelectric material such as quartz or the piezoelectric layer of an FBAR device. The resonator is used as the resonant element of an oscillator circuit. A receptor layer on a surface of the resonator provides sites for bonding to a target analyte that is desired to be detected. The oscillator oscillates at a frequency that depends on any mass associated with the resonator. The target analyte bonding to the receptor layer of the sensor changes the mass associated with the resonator and, hence, the resonant frequency of the oscillator. The sensor element is contacted with a fluid sample that may contain the target analyte. By measuring the frequency of the oscillator it can be determined whether the target analyte has bonded to the receptor layer and is therefore present in the sample.

Typically, sensors of the type just described are fabricated in arrays ranging from a few sensors to thousands of sensors in which the receptor layers of the sensors or of a group of the sensors are configured to bond with different analytes. Having multiple oscillators active at the same time produces significant interference effects that can impair the effectiveness of the detection. To avoid interference effects requires that the frequency of the oscillators be measured one at a time. However, this makes a detection process very slow and allows temporal effects such as temperature changes to impair the effectiveness of the detection. Moreover, sequential measurement prevents temporal relationships between different analytes in the sample from being observed. Also, temporal effects may give rise to significant error due to the uncertainty in calibrating the detection process.

Accordingly, what is needed is a mass sensor in which the mass loading of its constituent sensors can be simultaneously determined.

### BRIEF DESCRIPTION OF THE DRAWINGS

FIG. 1A is a block diagram showing an example of a mass sensor.

FIG. 1B is a block diagram showing an example of a mass sensor cell.

FIG. 2 is a block diagram showing an 102 of a system that incorporates an instance of a mass sensor for assaying a target analyte in a fluid sample.

FIG. 3A a schematic drawing showing an example of a mass-dependent RF filter.

FIG. 3B is a circuit diagram showing the equivalent circuit of an FBAR.

FIG. 4 is a graph showing the variation of the relative impedance of an FBAR with frequency.

FIG. 5 is a graph showing the frequency response of the example of the mass-dependent RF filter shown in FIG. 3A.

FIG. 6 shows cross-sectional views of exemplary FBAR embodiments that can be used to constitute the example of the mass-dependent RF filter shown in FIG. 3A.

FIG. 7 is a graph showing a portion of the frequency response of the example of the mass-dependent RF filter shown in FIG. 3A before and after receptors on the constituent FBARs have bonded with a target analyte.

FIGS. 8A-8C are graphs illustrating the time domain response of an example of the mass-dependent RF filter shown in FIG. 3A to a sine wave input signal.

FIG. 9 is a circuit diagram showing an example of a mass sensor cell.

FIGS. 10A-10C are graphs showing the time domain response of an example of the mass sensor cell shown in FIG. 9 to a sine wave input signal.

FIG. 11 is a circuit diagram showing an example of another embodiment of a hold circuit that holds a local output signal corresponding to the amplitude of the RF output signal output by the RF filter after the amplitude of the RF output signal has stabilized.

FIGS. 12A-12C are graphs showing the time domain response of an example of the mass sensor cell shown in FIG. 11 to a sine wave input signal.

### DETAILED DESCRIPTION

Disclosed herein is a mass sensor having a common RF input, a set of mass sensor cells, and a common hold input. Each of the mass sensor cells has a local input node coupled to the common RF input, a local output node, a mass-dependent RF filter that includes an electroacoustic resonator having receptors immobilized on a surface thereof, an RF detector, and a hold circuit having a local hold input. The RF filter, the RF detector and the hold circuit are connected in series between the local input node and the local output node. The common hold input is coupled to the local hold inputs of the hold circuits of at least a subset of the mass sensor cells.

In some embodiments, the electroacoustic resonator is a bulk acoustic wave (BAW) device, for example, a transverse-mode BAW device. In an example, the BAW device is a film bulk acoustic resonator (FBAR).

Also disclosed herein is a system for assaying a target analyte in a fluid sample. The system includes an RF oscillator, a mass sensor, an analog signal selector and an analog-to-digital converter. The mass sensor includes a common RF input coupled to the RF oscillator, a set of mass sensor cells and a common hold input. Each of the mass sensor cells includes a local input node coupled to the common RF input, a local output node, a mass-dependent RF filter, an RF detector and a hold circuit. The RF filter, the RF detector and the hold circuit are connected in series between the local input node and the local output node. The mass-dependent RF filter includes an electroacoustic resonator having receptors configured for bonding to the target analyte immobilized on a surface thereof. The hold circuit includes a local hold input. The common hold input is coupled to the local hold inputs of the hold circuits of a least a subset of the mass sensor cells. The analog signal selector includes a respective analog input connected to the output node of each of at least a subset of the mass sensor cells, a common analog output coupled to the analog-to-digital converter, and an address input to receive an address signal defining a specific one of the mass sensor cells whose output node the analog signal selector is to connect to the analog-to-digital converter.

Distributing an RF input signal from the common RF input to the local input nodes of all the mass sensor cells in parallel and sampling the outputs of the mass-dependent RF filters of all the mass sensor cells in parallel (by means of the common hold input) substantially eliminates differential temperature drift and/or differential temporal effects of different sensor reactions with the analyte between the mass sensor cells since no sequential sampling is needed.

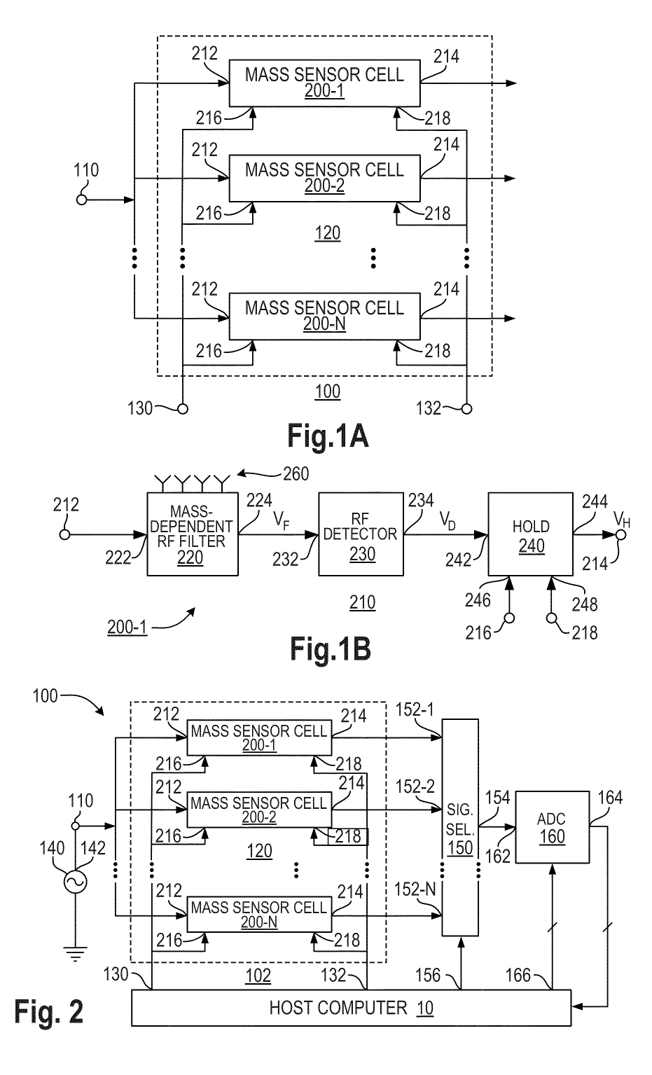

FIG. 1A is a block diagram showing an example 100 of a mass sensor. Mass sensor 100 includes a common RF input 110, a set 120 of mass sensor cells 200-1, 200-2, . . . , 200-N. Reference numeral 200 will be used to refer to a nonspecific one of the mass sensor cells or to the mass sensor cells in general. Mass sensor 100 additionally includes a common hold input 130 and a common reset input 132.

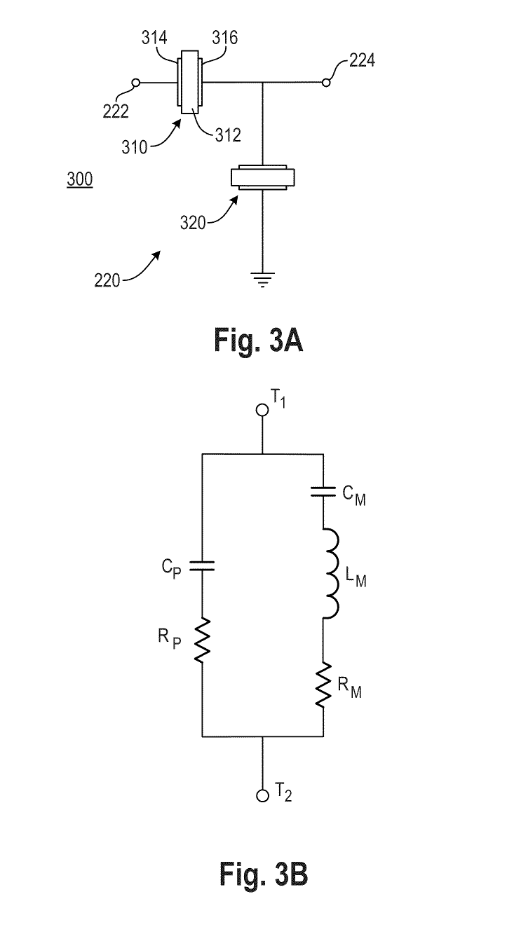

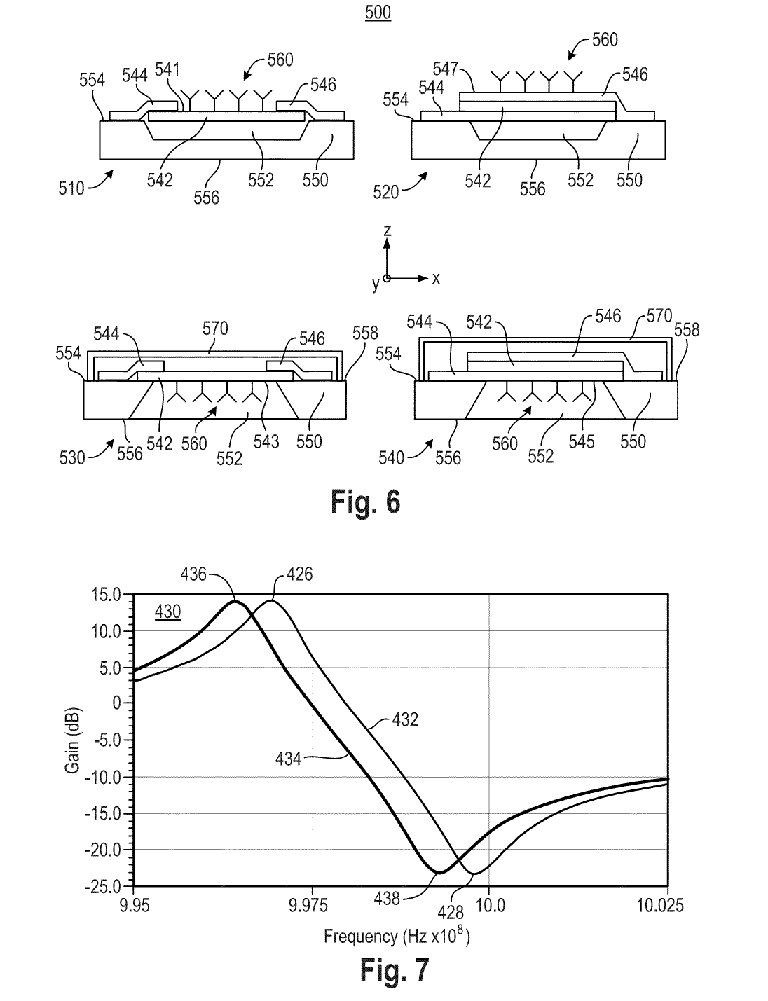

FIG. 1B is a block diagram showing an example 210 of mass sensor cell 200-1. Mass sensor cells 200-2, . . . 200-N are similar in structure and will not be separately described. Mass sensor cell 210 includes a local input node 212 coupled to common RF input 110, a local output node 214, a mass-dependent RF filter 220 that includes an electroacoustic resonator (not shown, but described below with reference to FIGS. 3A, 3B and 6) having receptors 260 immobilized on a surface thereof, an RF detector 230, and a hold circuit 240 having a local hold input 216 and a local reset input 218. RF filter 220, RF detector 230 and hold circuit 240 are connected in series between local input node 212 and local output node 214.

In the example shown, common RF input 110 is directly connected to the local input nodes 212 of mass sensor cells 200. In another example, an RF amplifier (not shown) is interposed between common RF input 110 and the local input nodes 212 of at least a subset of the mass sensor cells 200. The RF amplifier has sufficient output capacity to drive all the mass sensor cells connected to it.

The local hold inputs 216 of the hold circuits 240 of at least a subset of mass sensor cells 200 are coupled to common hold input 130. The local reset inputs 218 of the hold circuits 240 of at least a subset of mass sensor cells 200 are coupled to common reset input 132. In the example shown, the local hold inputs 216 and the local reset inputs 218 are directly connected to common hold input 130 and to common reset input 132, respectively. In other examples, suitable driver circuits are interposed between common hold input 130 and common reset input 132 and the local hold inputs 216 and the local reset inputs 218, respectively, of at least a subset of mass sensor cells 200.

Mass-dependent RF filter 220 is mass-dependent in the sense that it has a filter characteristic that depends on the mass of an analyte bonded to receptors 260. Mass-dependent RF filter 220 has an input 222 and an output 224. Input 222 is connected to local input node 212. RF detector 230 has an input 232 and an output 234. Input 232 is connected to the output 224 of RF filter 220. Hold circuit 240 has an input 242, an output 244, a hold input 246 and a reset input 248. Input 242 is connected to the output 234 of RF detector 230. Output 244 is connected to the local output node 214 of mass sensor cell 200. Hold input 246 is connected to the local hold input 216 of mass sensor cell 200. Reset input 248 is connected to the local reset input 218 of mass sensor cell 200.

FIG. 2 is a block diagram showing an example 102 of a system that incorporates an instance of mass sensor 100 for assaying a target analyte in a fluid sample. In addition to mass sensor 100, system 102 includes an RF oscillator 140, an analog signal selector 150, and an analog-to-digital converter (ADC) 160. In the example shown, system 102 additionally includes a controller 10.

RF oscillator 140 is an RF oscillator capable of generating an RF signal at the frequency of operation for which mass sensor 100 is designed. The stability of the frequency and amplitude of the RF signal generated by RF oscillator 140 should be sufficiently small that variations in the RF output signal V_{F} at the output 224 of mass-dependent RF filter 220 due to variations in the output of the RF oscillator are small compared with the change in RF output signal caused by the smallest change in mass loading that it is desired to detect. RF oscillator 140 has an RF output 142 connected to the common RF input 110 of mass sensor 100.

Analog signal selector 150 has a respective analog signal input 152-1, 152-2, . . . , 152-N connected to the local output node 214 of each of at least a subset of mass sensor cells 200-1, 200-2, . . . , 200-N. Reference numeral 152 will be used to refer to a nonspecific one of the analog signal inputs of analog signal selector 150, or to the analog signal inputs in general. Analog signal selector 150 additionally has a common analog output 154 and an address input 156. Address input 156 has conductors sufficient in number to receive a binary value equal to or greater than the binary equivalent of the number N of mass sensor cells 200. ADC 160 has an analog input 162, a digital output 164 and a control input 166. Analog input 162 is connected to the common analog output 154 of analog signal selector 150.

Common hold input 130 and common reset input 132 of mass sensor 100 are connected to receive a hold signal and a reset signal from controller 10. The address input 156 of analog signal selector 150 is connected to receive address signals from controller 10. The digital output 164 of ADC 160 is connected to provide numerical values to an input of controller 10. Controller 10 stores the numerical values generated by ADC 160 for each of the mass sensor cells 200, and subjects numerical values received from ADC 160 to arithmetic operations, as will be described below. In some embodiments, an external device capable of generating control signals and address signals and of receiving and storing numerical values and subjecting such numerical values to arithmetic operations may be substituted for controller 10. In other embodiments, controller 10 only generates control signals and address signals, and an external device receives and stores numerical values and subjects such numerical values to the arithmetic operations described below is being performed by controller 10.

The example of system 102 shown has a single analog signal selector 150 in which a respective analog signal input 152 of the analog signal selector is connected to the local output node 214 of each of all the mass sensor cells 200 constituting mass sensor 100. Other examples have more than one analog signal selector in which each of the analog signal selectors has a respective analog signal input 152 connected to the local output node of each of a subset of the mass sensor cells 200 constituting mass sensor 100. In some examples, the analog output of each analog signal selector is connected to the analog input of a respective ADC. The digital outputs of the ADCs are then multiplexed prior to input into controller 10. In other examples, the analog outputs of the analog signal selectors are multiplexed using additional analog signal selectors arranged in one or more hierarchical layers. The analog output of a final analog signal selector in the hierarchy is connected to the analog input of ADC 160. When multiple analog signal selectors are used, each analog signal selector receives at his address input 156 a range of address signals corresponding to the mass sensor cells 200 to which the analog signal selector is connected. In an example in which mass sensor cells are arranged in an array of rows and columns, the inputs of a respective first-level analog signal selector are connected to the mass sensor cells in each row (or part of each row) of the array, and the inputs of one or more second-level analog signal selectors are connected to the outputs of the first-level analog signal selectors.

Typically, mass sensor 100 is one of a few up to thousands of mass sensors fabricated by subjecting one or more semiconductor or ceramic wafers to a series of fabrication operations, such as photolithography, etching, deposition, and passivation, to fabricate the circuitry and the electroacoustic resonators that constitute each mass sensor. Some processes fabricate the electroacoustic resonators and the circuitry of mass sensor 100 on a common wafer. Other processes fabricate the electroacoustic resonators on one wafer and the circuitry of mass sensor 100 on another wafer. The wafers are then joined together to form a single wafer. The wafer is then singulated into individual die, each of which typically embodies one instance of mass sensor 100. In typical but not all embodiments, to ameliorate the problem of outputting from mass sensor 100 the local output signals V_{H} output at the local output nodes 214 of what may be thousands of mass sensor cells 200, analog signal selector 150 is additionally fabricated on the same die as the circuitry of the mass sensor. In some embodiments, one or both of RF oscillator 140 and ADC 160 are also fabricated on the same die as the circuitry of mass sensor 100. In other embodiments, one or more of RF oscillator 140, analog signal selector 150 and ADC 160 are external components connected to mass sensor 100. Controller 10 is typically an external component connected to mass sensor 100. However, in some embodiments, additional circuitry is fabricated on the same die as mass sensor 100 to provide the control signal generation, storage and arithmetic functionalities of controller 10.

Operation of mass sensor 100 and system 102 involves using mass sensor 100 to make at least two sets of measurements. The first set of measurements, referred to herein as a pre-contacting set of measurements, is made before mass sensor 100 is contacted with the sample. A second set of measurements, and possibly subsequent sets of measurements, are made at defined times after mass sensor 100 has been contacted with the sample. Each set of measurements made after mass sensor 100 has been contacted with a sample are referred to herein as a post-contacting set of measurements.

Prior to making the pre-contacting set of measurements, RF oscillator 140 is turned on for a time sufficient to allow the oscillator frequency and amplitude to stabilize. Controller 10 then initializes mass sensor 100 by asserting a hold signal at common hold input 130 and a reset signal at common reset input 132. Asserting the hold signal essentially disconnects hold circuit 240 from RF detector 230. Asserting the reset signal resets the local output signal at the output 244 of the respective hold circuits 240 of all the mass sensor cells 200 to zero.

In response to the RF signal received at its input 222 from RF oscillator 140, the RF filter 220 of each mass sensor cell 200 outputs at its output 224 an RF output signal V_{F} whose amplitude and phase depend on the amplitude and frequency of the RF input signal received at input 222 from RF oscillator 140, the filter characteristic of the RF filter, and the load imposed on the filter by the RF detector and subsequent circuitry. Each RF detector 230 converts signal V_{F} received at its input 232 from its respective RF filter 220 to a detection signal V_{D} that depends on signal V_{F}. In an example, detection signal V_{D} depends on the peak, RMS, average or mean amplitude of RF output signal V_{F}. In another example, detection signal V_{D} depends on the phase of RF output signal V_{F}. The detection signal V_{D} generated by each RF detector 230 is output at output 234 and is received at the input 242 of its respective hold circuit 240. However, since the hold circuit is in its reset state, the local output signal V_{H} at the output 244 of the hold circuit remains at zero.

Controller 10 then applies to mass sensor 100 a sequence of control signals and address signals that control the generation of a set of pre-contacting numerical values. Each pre-contacting numerical value represents the local output signal V_{H} at the local output node 214 of a respective one of the mass sensor cells 200 constituting mass sensor 100 before the mass sensor is contacted with a sample. Each local output signal V_{H}, in turn, represents a property (e.g., amplitude or phase) of the RF output signal V_{F} at the output 224 of the RF filter 220 of the one of the mass sensor cells 200.

At the start of the sequence, the controller de-asserts the hold signal at common hold input 130 and the reset signal at common reset input 132. This allows the local output signal V_{H} at the output 244 of each hold circuit 240, and, hence, the local output signal V_{H} at the respective local output node 214, to increase to a level substantially equal to the respective detection signal V_{D} at the input 242 of the hold circuit. After a defined integration time, the controller reasserts the hold signal at common hold input 130. The reasserted hold signal causes the respective hold circuit 240 of each mass sensor cell 200 to hold local output signal at its output 244 and, hence, local output node 214, at a level substantially equal to its level at the instant the hold signal was reasserted. A longer integration time between de-asserting the hold signal and reasserting the hold signal can be used to reduce noise on the local output signal V_{H} output by the hold circuit.

Analog signal selector 150 receives at its analog signal inputs 152 a set of local output signals V_{H} output at the local output nodes 214 of mass sensor cells 200. Each local output signal is held at a level equal to its level at the instant the hold signal was reasserted. Controller 10 then provides a sequence of address signals to analog signal selector 150. Each address signal in the sequence defines the address of a respective one of the analog signal inputs 152 of the analog signal selector and causes the analog signal selector to output at its common analog output 154 the local output signal received at the analog signal input 152 selected in response to the address signal. The controller additionally provides a control signal to the control input 166 of ADC 160. The control signal causes the ADC to convert the local output signal received at its analog input 162 to a pre-contacting numerical value that the ADC outputs to the controller via its digital output 164. The controller stores each pre-contacting numerical value received from ADC 160 linked to the address indicated by the corresponding address signal output by the controller to analog signal selector 150.

Once controller 10 has stored numerical values representing the local output signals V_{H} at the local output nodes 214 of all of the mass sensor cells 200 constituting mass sensor 100, the host controller reasserts the reset signal at common reset input 132. This sets the local output signals at the local output nodes 214 of all the mass sensor cells 200 to 0 V relative to ground, or to another predetermined level.

Mass sensor 100 is then contacted by the sample (not shown). Once the mass sensor has been contacted by the sample, the controller applies to mass sensor 100 the above-described sequence of control signals and address signals that control the generation of a set of post-contacting numerical values. Each post-contacting numerical value represents local output signal V_{H} at the local output node 214 of a respective one of the mass sensor cells 200 constituting mass sensor 100 at a defined time after the mass sensor is contacted with the sample. Each local output signal V_{H}, in turn, represents a property (e.g., amplitude or phase) of the RF output signal V_{F} of the RF filter 220 of the one of the mass sensor cells 200.

Contacting mass sensor 100 with the sample typically results in the receptors 260 on the RF filter 220 of one or more of the mass sensor cells 200 binding with a respective target analyte in the sample. The target analyte bound to the receptors 260 increases the mass loading of the respective RF filter and produces a corresponding change in the filter characteristics of the RF filter. As a result, the property of the RF output signal V_{F} of the RF filter represented by the local output signal V_{H} at the local output node 214 of the mass sensor cell, and the corresponding post-contacting numerical value output by ADC 160 differ from the corresponding pre-contacting values of these parameters.

Controller 10 subtracts each post-contacting numerical value it receives from ADC 160 from the corresponding pre-contacting numerical value it has stored linked to the address indicated by the same address signal output by the controller to analog signal selector 150 to generate a respective difference value. Alternatively, controller 10 stores each post-contacting numerical value received from ADC 160 linked to the address indicated by the corresponding address signal output by the controller to analog signal selector 150 and subsequently performs the above-described subtraction using the stored pre-contacting numerical value and the stored post-contacting numerical value to generate a respective difference value. The difference value represents the difference between the pre-contacting numerical value and the post-contacting numerical value for each mass sensor cell 200 represents the mass of the target analyte (if any) bonded to the receptors 260 of the RF filter 220 of the mass sensor cell.

In some embodiments, controller 10 additionally stores data indicating a target analyte corresponding to the receptors 260 on each of the mass sensor cells 200. In such embodiments, controller 10 additionally performs processing to display a list of target analytes having a calculated difference value greater than zero (or another threshold difference) and, for each such target analyte, a corresponding concentration of the target analyte in the sample. The controller calculates the concentration of the target analyte in the sample from the difference value calculated for the mass sensor cell having immobilized on a surface thereof receptors that bind to the target analyte. The relationship between target analyte concentration and calculated difference value is obtained by calculating difference values obtained using samples having known concentrations of the target analyte.

In some embodiments, after the set of post-contacting numerical values has been generated, controller 10 again reasserts the reset signal at common reset input 132 and then, after a defined time interval, applies to mass sensor 100 the above-described sequence of control signals and address signals to generate an additional set of post-contacting numerical values, and an additional set of calculated difference values. The additional set of calculated difference values is constituted of difference values calculated between the set of pre-contacting numerical values and the additional set of post-contacting numerical values. Several sets of post-contacting numerical values and corresponding sets of calculated difference values can be generated to quantify a rate of binding between the target analyte and the receptors.

Mass sensor 100 will now be described in greater detail with reference to FIGS. 3A, 3B, and 4-12. FIG. 3A a schematic drawing showing an example 300 of mass-dependent RF filter 220. In the example shown, mass-dependent RF filter 300 includes a series film bulk acoustic resonator (FBAR) 310 connected between input 222 and output 224 and a shunt FBAR 320 connected between output 224 and ground 226. “Ground” in the context of mass-dependent RF filter 220 is signal ground. An FBAR is a type of bulk acoustic wave (BAW) device. Other types of BAW device may be used in mass-dependent RF filter 220. A BAW device is a type of electroacoustic resonator. Other types of electroacoustic resonator may be used in mass-dependent RF filter 220.

Series FBAR 310 includes a piezoelectric layer 312 and a pair of electrodes 314, 316 electrically coupled to piezoelectric layer 312. Piezoelectric layer 312 is a layer of a piezoelectric material, such as aluminum nitride (AlN) or zinc oxide (ZnO), that converts an alternating electrical signal applied between electrodes 314, 316 to mechanical vibrations of the piezoelectric layer. The mechanical vibrations of piezoelectric layer 312 cause the piezoelectric layer to generate an alternating electrical signal between electrodes 314, 316.

FIG. 3B is a circuit diagram showing the equivalent circuit of series FBAR 310. The equivalent circuit of series FBAR 310 includes what will be referred to herein as a motional capacitance C_{M}, a motional inductance L_{M} and a motional resistance R_{M} connected in series to form a first branch, and a shunt capacitance C_{P} and a shunt resistance R_{P} connected in series to form a second branch. The second branch is connected in parallel with the first branch between terminals T₁ and T₂. Shunt capacitance C_{P} is the capacitance of a capacitor formed by electrodes 314, 316 and piezoelectric layer 312. Shunt resistance R_{P} is the series resistance of shunt capacitance C_{P}. Motional inductance L_{M}, motional capacitance C_{M} and motional resistance R_{M} respectively represent an inductance, a capacitance and a resistance that originate from the mechanical properties of series FBAR 310. Specifically, motional inductance L_{M} depends on the mass of series FBAR 310, motional capacitance C_{M} depends on the Young's modulus of the FBAR, and motional resistance R_{M} represents mechanical losses in the series FBAR.

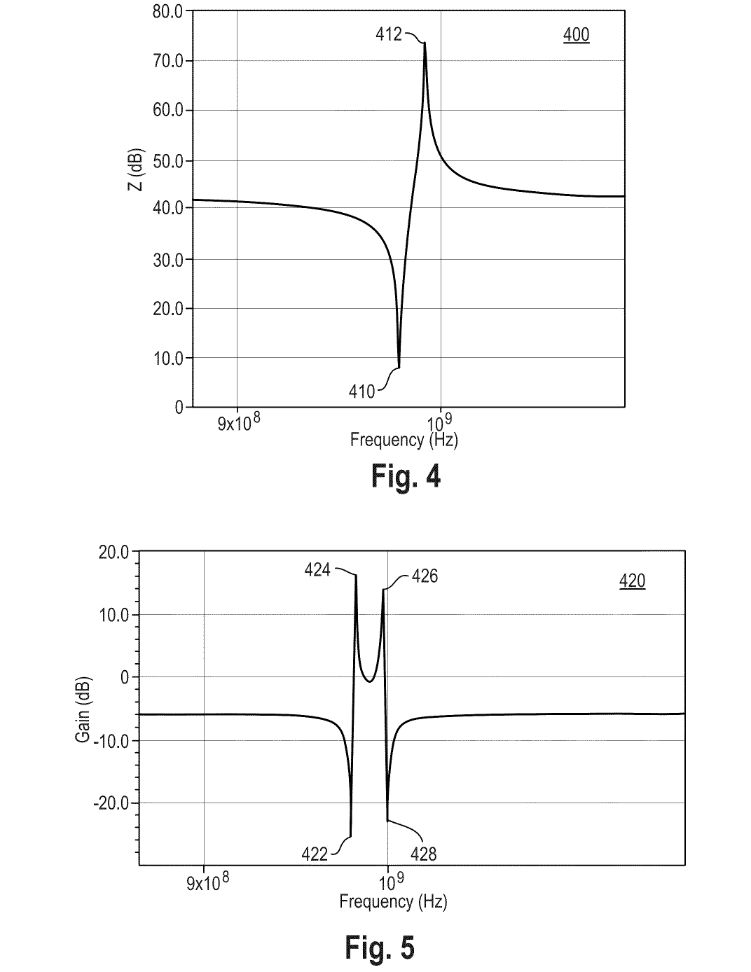

FIG. 4 is a graph 400 showing the variation of the relative impedance of series FBAR 310 between terminals T₁ and T₂ with frequency. The x-axis of the graph shows frequency on a logarithmic scale in a frequency range from about 870 MHz to about 1.1 GHz. The y-axis shows the impedance of series FBAR 310 expressed in decibels. Initially, as the frequency increases, the impedance of series FBAR 310 gradually falls due to the falling impedance of the shunt capacitance C_{P}. As the frequency approaches the frequency of the series resonance between motional inductance L_{M} and motional capacitance C_{M}, the impedance falls sharply to a minimum 410 at the frequency of the series resonance. As the frequency increases above the frequency of the series resonance, the impedance sharply increases to a maximum 412 at the frequency of the parallel resonance between motional inductance L_{M} and the series combination of motional capacitance C_{M} and shunt capacitance C_{P}. Since shunt capacitance C_{P} is about 20 times larger than motional capacitance C_{M}, the frequency difference between the series resonance and the parallel resonance is small. The impedance then falls steeply as the frequency increases above the frequency of the parallel resonance.

Shunt FBAR 320 is similar in structure and operation to series FBAR 310. However, shunt FBAR 320 differs slightly in mass from series FBAR 310 so that the resonant frequencies of shunt FBAR 320 differ from those of series FBAR 310. In an example, the mass of shunt FBAR 320 is slightly more than that of series FBAR 310 so that the series resonance of shunt FBAR 320 is at a lower frequency than that of series FBAR 310. The difference in mass is typically accomplished by making at least one of the electrodes of shunt FBAR 320 larger in area or thicker than, or both larger in area and thicker than, corresponding electrodes of series FBAR 310. Other ways of changing the mass of an FBAR are known and may be used.

FIG. 5 is a graph 420 showing the frequency response of the example 300 of mass-dependent RF filter 220 (FIG. 1B) described above with reference to FIGS. 3A and 3B. The example is configured to operate at a frequency of about 1 GHz. The x-axis of the graph shows frequency on a logarithmic scale in a frequency range from about 870 MHz to about 1.1 GHz. The y-axis shows the gain (20× log(abs(Voutput/Vinput))) of exemplary RF filter 300 expressed in decibels. The response of exemplary RF filter 300 exhibits a lower-frequency minimum 422 generated by the series resonance of shunt FBAR 320, a lower-frequency maximum 424 generated by the series resonance of series FBAR 310, a higher-frequency maximum 426 generated by the parallel resonance of the shunt FBAR 320 and a higher-frequency minimum 428 generated by the parallel resonance of series FBAR 310.

Other embodiments of RF filter 220 have a structure similar to RF filter 300, but series FBAR 310 is replaced by a transconductance element, (not shown), e.g., a transconductance amplifier, that outputs a current dependent on the RF input signal received at input 222. In such an embodiment, the frequency of the RF input signal is within a frequency range that extends from below to above the frequency of maximum impedance 412 of the impedance characteristic (FIG. 4) of shunt FBAR 320 where the impedance characteristic is steeply sloped. However, the steepness of the slope of the impedance characteristic, and a combination of dynamic range and steepness of slope of a single FBAR implementation are inferior to those of the two FBAR implementation described above with reference to FIG. 3A.

FIG. 6 shows cross-sectional views of various exemplary FBAR embodiments 510, 520, 530, 540 that can be used as FBARs 310, 320 of exemplary mass-dependent RF filter 300 (FIG. 3A). Corresponding elements of the different embodiments are indicated using the same reference numeral and will not be repetitively described. Reference numeral 500 will be used to refer to FBARs 510, 520, 530, 540 when common features are described. FBAR 500 includes a piezoelectric layer 542 and pair of electrodes 544, 546 electrically coupled to piezoelectric layer 542.

FBAR 500 is suspended over a cavity 552 defined in a substrate 550. In some embodiments, each FBAR 500 is suspended over a respective cavity 552. In other embodiments, two or more FBARs 500 are suspended over a common cavity 552. Suspending FBAR 500 over cavity 552 allows the FBAR to resonate mechanically in response to an alternating electrical signal applied between its electrodes. Other suspension schemes that allow FBAR 500 to resonate mechanically are possible. In an example applicable to FBARs 510, 520, the FBAR is a solidly-mounted FBAR that is acoustically isolated from substrate 550 by an acoustic Bragg reflector (not shown), such as that described by John D. Larson III et al. in U.S. Pat. No. 7,332,985 entitled Cavity-Less Film Bulk Acoustic Resonator (FBAR) Devices.

In FBARs 510, 530, electrodes 544, 546 electrically contact piezoelectric layer 542 at locations offset from one another in the x-direction, parallel to the major surface 556 of substrate 550. An alternating electrical signal applied between electrodes 544, 546 causes the FBAR to vibrate in the x-direction. Shear-mode FBARs such as FBARs 510, 530 are suitable for use with liquid or gaseous samples.

In FBARs 520, 540, piezoelectric layer 542 is sandwiched between electrodes 544, 546 so that electrodes 544, 546 electrically contact piezoelectric layer 542 at locations offset from one another in the z-direction, orthogonal to the surface 556 of substrate 550. An alternating electrical signal between electrodes 544, 546 causes the FBAR to vibrate in the z-direction at the frequency of the electrical signal. Longitudinal-mode FBAR such as FBARs 520, 540 are suitable for use with gaseous samples. The quality factor (Q) of longitudinal-mode FBARs is reduced when used with samples because the liquid damps the longitudinal wave in the FBAR resulting in lower sensitivity for liquid samples.]

FBAR 500 additionally includes receptors 560 immobilized on a major surface thereof. In FBAR 510, receptors 560 are immobilized on the major surface 541 of piezoelectric layer 542 remote from substrate 550. In FBAR 520, receptors 560 are immobilized on the major surface 547 of electrode 546 remote from substrate 550. Receptors 560 are contacted by a sample (not shown) flowing along the side 554 of substrate 550 that supports FBARs 500.

In FBAR 530, receptors 560 are immobilized on the major surface 543 of piezoelectric layer 542 facing substrate 550. In FBAR 540, receptors 560 are immobilized on the major surface 545 of electrode 544 facing substrate 550. In FBARs 530 and 540, cavity 552 extends through the thickness of substrate 550 to provide access to receptors 560 from the side 556 of substrate 550 remote from FBAR 500. Receptors 560 are contacted by a sample (not shown) flowing along the side 556 of substrate 550. FBARs 530, 540 additionally include a cap 570 of the side 558 of substrate 550. Cap 570 covers the FBAR to prevent the sample from contacting both of electrodes 544, 546 and potentially short-circuiting them. Other ways of passivating at least one of electrodes 544, 546 to prevent short-circuiting are known and may be used. Typically, a common cap 570 is used to cover the FBARs of the mass-dependent RF filters 220 of all, or a subset of, the mass sensor cells 200 and of mass sensor 100. In some implementations, a semiconductor die on which at least part of the circuitry of mass sensor 100 is fabricated serves as common cap 570.

Receptors 560 are any type of receptor that will bond with a target analyte of interest. Examples of receptors that may be used as receptors 560 include, but are not limited to, nucleic acids (e.g., strands of DNA or RNA), antibodies, enzymes, and other receptors that will bond with bomb materials, pollutants, harmful gases in air or water, disease agents, etc. Receptors 560 are immobilized on the major surface of FBARs 500 by any suitable means. In an example, antibodies are attached to FBAR 500 by covalent attachment by conjugation of amino, carboxyl, aldehyde, or sulfhydryl groups. Prior to attaching the receptors, the major surface of the FBAR on which the receptors are to be immobilized is functionalized with an amino, carboxyl, hydroxyl, or other group.

Contacting an FBAR 500 with a sample that contains a target analyte capable of bonding with receptors 560 causes the analyte to bond with some or all of the receptors. The target analyte bonded to receptors 560 increases the mass loading of the FBAR. Since the series and parallel resonant frequencies of the FBAR depend on the mechanical inductance of the FBAR and, hence, on the mass loading of the FBAR, the analyte bonded to receptors 560 decreases the resonant frequencies of the FBAR by a frequency difference that depends on the quantity of target analyte bonded to receptors 560.

FIG. 7 is a graph 430 showing a portion of the frequency response of the example 300 of mass-dependent RF filter 220 (FIG. 1) described above with reference to FIGS. 3A and 3B before (trace 432) and after (trace 434) an increase of about 0.1% in the motional inductance of FBARs 310, 320. This increase in the motional inductance simulates the increase in the mass loading of the FBARs of mass-dependent RF filter 220 due to the bonding of a quantity of a target analyte to receptors 260. Graph 430 is similar to graph 420 shown in FIG. 5, but the frequency scale on the x-axis is substantially expanded, and only a portion of the response between the higher-frequency maximum 426 generated by the parallel resonance of the shunt FBAR 320 and the higher-frequency minimum 428 generated by the parallel resonance of series FBAR 310 is shown. Trace 432 indicates the frequency response of mass-dependent RF filter 220 before receptors 260 immobilized on a surface of FBARs 310, 320 have bonded with a target analyte. The response indicated by trace 432 is the same as that shown in FIG. 5. Trace 434 indicates the frequency response of mass-dependent RF filter 220 after receptors 260 have bonded with the target analyte. The response indicated by trace 434 exhibits a higher-frequency maximum 436 generated by the parallel resonance of the shunt FBAR 320 and a higher-frequency minimum 438 generated by the parallel resonance of series FBAR 310. The additional mass of FBARs 310, 320 after the target analyte has bonded to receptors 260 reduces the frequencies of higher-frequency maximum 436 and higher-frequency minimum 438 relative to higher-frequency maximum 426 and higher-frequency minimum 428.

It can be seen from FIG. 7 that, at any fixed frequency between higher-frequency maximum 426 and higher-frequency minimum 438, e.g., 997.5 MHz, the target analyte bonded to receptors 260 on FBAR 310, 320 reduces the amplitude of the RF output signal V_{F} output by RF filter 220 by about 6 dB. Accordingly, comparing the amplitude of output signal V_{F} output by RF filter 220 before and after receptors 260 on FBARs 310, 320 have been exposed to the sample provides a measure of the quantity of the target analyte bonded to receptors 260. Such a measure can be used to determine the presence or absence of the target analyte in the sample. Additionally, since the reduction in output signal V_{F} depends on the additional mass of the target analyte bonded to receptors 260, with at least some target analyte/receptor combinations, the measure can be used to determine the concentration of the target analyte in the sample.

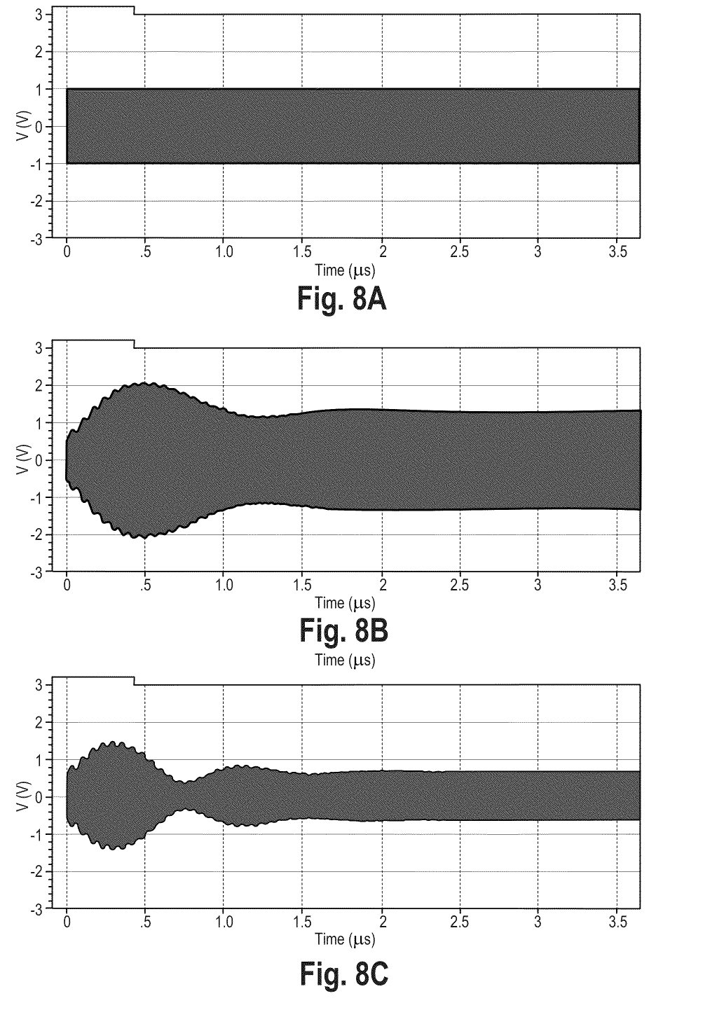

FIGS. 8A-8C are graphs illustrating the time domain response of an example of mass-dependent RF filter 300 (FIG. 3A) to a sine wave input signal over a time span of 3.5 μs. FIG. 8A shows the envelope of a 997.5 MHz input signal with an amplitude of 2V peak to peak applied to input 222 of mass-dependent RF filter 300 shown in FIG. 2. FIG. 8B shows the envelope of the RF output signal V_{F} output by mass-dependent RF filter 300 at output 224 prior to a change in the motional inductance of FBARs 310, 320 (FIG. 3A). The amplitude of RF output signal V_{F} stabilizes at about 2.6 V peak to peak. FIG. 8C shows the envelope of RF output signal V_{F} after a 0.1% increase in the motional inductance of FBARs 310, 320. This is the same increase in motional inductance that produced the change in the filter response shown in FIG. 7. The amplitude of RF output signal V_{F} stabilizes at about 1.2 V peak to peak.

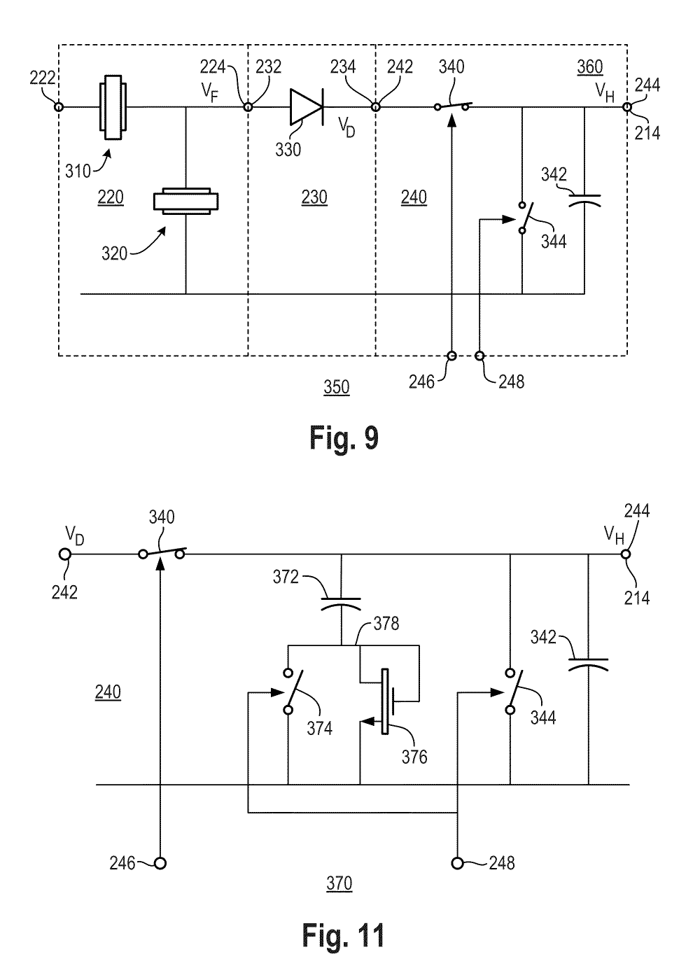

FIG. 9 is a circuit diagram showing an example 350 of mass sensor cell 200-1 described above with reference to FIG. 1B. Elements of exemplary mass sensor cell 350 described above with reference to FIG. 1B are indicated using the same reference numerals and will not be described again in detail. Mass-dependent RF filter 220 is implemented using the RF filter example 300 described above with reference to FIGS. 3A and 3B. RF detector 230 is implemented using a diode 330. The anode of diode 330 is connected to input 232 and the cathode of diode 330 is connected to output 234. In another example, the polarity of diode 330 is the opposite of that shown. Other circuits are known that generate a local output signal representative of the peak, RMS, mean, or average amplitude or representative of the phase of an alternating signal and may be used as RF detector 230. In some embodiments, RF detector 230 additionally includes a shunt capacitor (not shown) connected between the cathode of diode 330 and ground 226 to provide RF detector 230 with an integrating characteristic. RF detector 230 may additionally include one or both of a series resistor and a shunt resistor (not shown) to further define the integrating characteristic of the RF detector. The integrating characteristic of RF detector 230 may be in addition to or instead of any integrating characteristic of hold circuit 240.

Using diode 330 as RF detector 230 results in an offset in the local output signal V_{H} output output by mass sensor cell 350. The offset may be changed electrically between the output of mass sensor cell 350 and ADC 160. Alternatively, different configurations of RF detector 230 and hold circuit 240 having different offsets and/or different dynamic ranges can be used to address the offset issue or to change the dynamic range electronically, if desired. The offset may be addressed and/or the dynamic range may be changed individually in each sensor cell or, more efficiently, once at the input of ADC 160.

The example 360 of hold circuit 240 shown is a track-and-hold circuit that includes a hold switch 340, a capacitor 342 and a reset switch 344. A track and hold circuit integrates detection signal V_{D} output by RF detector 230, which reduces noise on local output signal V_{H} output by hold circuit 240. Hold switch 340 is connected between input 242 and output 244. Reset switch 344 is connected in parallel with capacitor 342 and the parallel combination is connected between output 244 and ground 226. Hold switch 340 and reset switched 344 are controlled switches having respective control inputs. The control input of hold switch 340 is connected to hold input 246 that in turn is connected to the common hold input 130 (FIG. 1A) of mass sensor 100. The control input of reset switch 344 is connected to reset input 248 that in turn is connected to the common reset input 132 of mass sensor 100. Some embodiments include a buffer (not shown) having an input connected to the node at which hold switch 340, reset switch 344 and capacitor 342 interconnect and an output connected to the output 244 of hold circuit 240. The buffer has a high input impedance to reduce the rate at which the voltage held on capacitor 340 decays.

Initially, the hold signal at hold input 246 is asserted, so that hold switch 340 is open, and the reset signal received at reset input 248 is asserted, so that reset switch 344 is closed. Reset switch 344 in its closed state maintains capacitor 342 in a discharged state so that the local output signal V_{H} at the output 244 of hold circuit 240, and the local output signal V_{H} at the local output node 214 of mass sensor cell 350 are both at 0 V relative to ground. After the output of RF oscillator 140 (FIG. 2) has stabilized in frequency and amplitude, the hold signal at hold input 246 is de-asserted, which causes hold switch 340 to connect the output 234 of RF detector 230 to capacitor 342. Additionally, the reset signal at reset input 248 is de-asserted, which causes reset switch 344 to disconnect output 244 from ground 226. Current from RF detector 230 progressively charges capacitor 342 to a voltage corresponding to the peak amplitude of the RF output signal V_{F} of mass-dependent RF filter 220.

It should be noted that, during the charging transient of hold circuit 240, the loading imposed by RF detector 230 and hold circuit 240 changes the characteristics of RF filter 220 relative to the example of RF filter 300 whose response is described above with reference to FIGS. 8A-8C. At the end of the charging transient, RF detector 230 allows RF filter 220 to return to its original filter characteristic because the loading effects are significantly reduced as diode 330 turns off as hold capacitor 342 reaches final value.

After a defined integration time, the hold signal at hold input 246 is re-asserted, which causes hold switch 340 to a disconnect capacitor 342 from the output 234 of RF detector 230. Capacitor 342 retains the voltage thereon until such time as it is discharged by the assertion of the reset signal at reset input 248 causing reset switch 344 to close. During the time that capacitor 342 retains the voltage thereon, the local output signal V₁ at the local output node 214 of mass sensor cell 350 is selected by analog signal selector 150 and is converted to a numerical value by ADC 160, as described above with reference to FIG. 2.

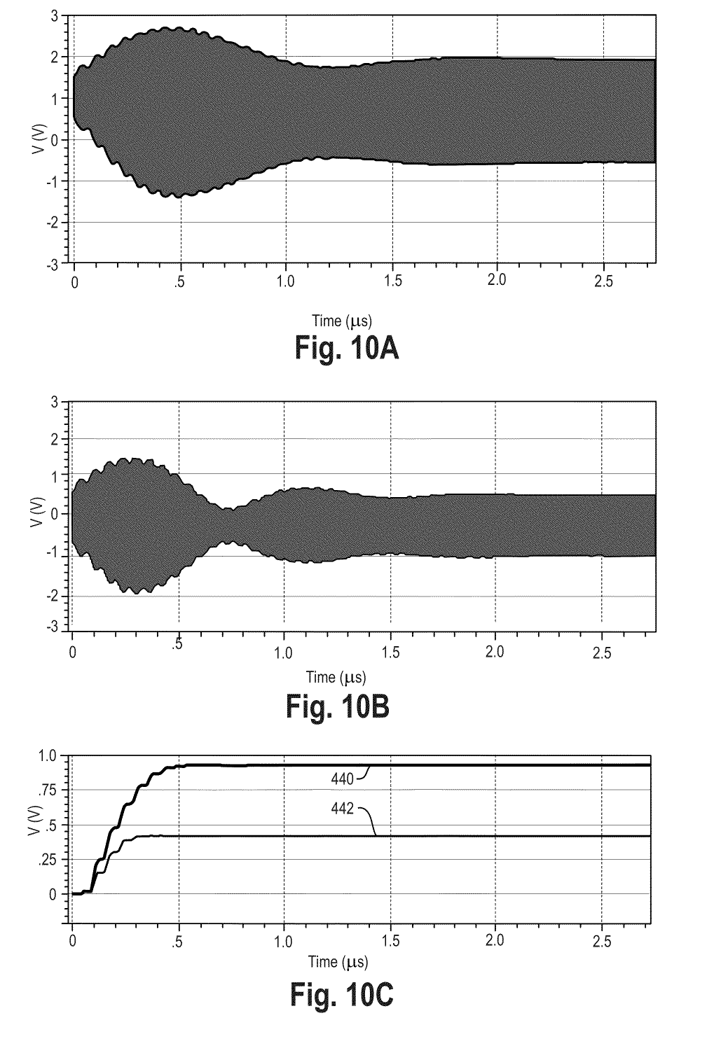

FIGS. 10A-10C are graphs showing the time domain response of an example of mass sensor cell 350 to a sine wave input signal over a time span of 3 μs. In the example shown, hold switch 340 is closed and reset switch 344 is open. The envelope of a 997.5 MHz input signal with an amplitude of 2V peak-to-peak applied to input 222 of mass sensor cell 350 is similar to that shown in FIG. 8A. FIG. 10A shows the envelope of the RF output signal V_{F} output by mass-dependent RF filter 220 at output 224 prior to an increase in the motinal inductance of FBARs 310, 320 (FIG. 3A). The asymmetry exhibited by the waveform is due to the loading imposed on the output of RF filter 220 by capacitor 342 in the positive half cycles during which diode 330 conducts. Once capacitor 342 is charged, the amplitude of the positive half cycles of the output of RF filter 220 stabilizes at about 0.9V. FIG. 10C shows the local output signal V_{H} at the local output node 214 of mass sensor cell 350. In FIG. 10C, trace 440 shows the local output signal V_{H} at local output node 214 prior to the increase in the motional inductance of FBARs 310, 320, local output signal V_{H} stabilizes at about 0.9 V. This is approximately one diode drop below the peak amplitude (approximately 1.7 V) of the positive half cycles of RF output signal V_{F}. The peak amplitude is attained approximately 0.5 μs after the RF input signal is applied to input 222.

FIG. 10B shows the envelope of RF output signal V_{F} output by mass-dependent RF filter 220 at output 224 after a 0.1% increase in the motional inductance of FBARs 310, 320 (FIG. 3A). This is the same increase in motional inductance as that which produced the change in the response of mass-dependent RF filter 220 shown in FIG. 7. The asymmetry in the waveform is again due to the loading imposed on RF filter 220 by capacitor 342 as diode 330 conducts during positive half cycles of RF output signal V_{F}. In FIG. 10C, trace 442 shows the local output signal V_{H} at local output node 214 after the increase in the motional inductance of FBARs 310, 320, local output signal V_{H} stabilizes at about 0.45 V. This is approximately one diode drop below the peak amplitude (approximately 1.25 V) of the positive half cycles of RF output signal V_{F}. The peak amplitude is attained less than 0.5 μs after the RF input signal is applied to input 222. Thus, a 0.1% increase in the motional inductance of FBARs 310, 320 produces a substantial change in the local output signal V_{H} output by mass sensor cell 350.

The example of mass sensor cell 350 described above with reference to FIGS. 9 and 10A-10C has a peak-reading characteristic so that the local output signal V_{H} at output at local output node 214 depends on the peak amplitude of the RF output signal V_{F} at the output 224 of mass-dependent RF filter 220. As noted above, RF output signal V_{F} attains its peak amplitude about 0.5 μs after the RF input signal is applied to the input of the RF filter. This is well before the amplitude of RF output signal V_{F} stabilizes. A more accurate measure of the increase in mass loading of mass-dependent RF filter 220 is obtained when hold circuit 240 holds the local output signal at a level corresponding to the amplitude of RF output signal V_{F} after the amplitude of the RF output signal has stabilized, compared with when hold circuit 240 holds a DC level corresponding to the peak amplitude of the RF output signal. Holding the local output signal at the level corresponding to the amplitude of the RF output signal after the amplitude of the RF output signal has stabilized provides an averaging effect that can filter out higher frequency noise, for example.

FIG. 11 is a circuit diagram showing an example 370 of another embodiment of hold circuit 240 that holds a local output signal V_{H} corresponding to the amplitude of the RF output signal V_{F} output by RF filter 220 at output 224 after the amplitude of RF output signal V_{F} has stabilized. In addition to the circuit elements of hold circuit 360 of the exemplary mass sensor cell 350 described above with reference to FIG. 9, hold circuit 370 includes a capacitor 372, an initialization switch 374 and a transistor 376. Initialization switch 374 is connected in parallel with the current path of transistor 376 between source and drain and the parallel combination is connected in series with capacitor 372. The series/parallel combination is connected between ground 226 and the node between hold switch 340 and output 244. Capacitor 372, initialization switch 374, and the gate and drain of transistor 376 are interconnected at a node 378. The capacitance of capacitor 372 and the size and threshold voltage of transistor 376 are appropriately chosen to determine the usable dynamic range. The control input of initialization switch 374 is connected to reset input 248. With this arrangement, the reset signal asserted at reset input 248 closes both reset switch 344 and initialization switch 374.

Referring additionally to FIG. 11, initially, the hold signal at hold input 246 is asserted, so that hold switch 340 is open, and the reset signal at reset input 248 is asserted, so that reset switch 344 and initialization switch 374 are both closed. Reset switch 344 in its closed state maintains capacitor 342 in a discharged state so that the local output signal at the output 244 of hold circuit 370, and the local output signal at the local output node 214 of mass sensor cell 350 are both at 0 V relative to ground. Initialization switch 374 in its closed state maintains capacitor 372 in a discharged state since both terminals of capacitor 372 are connected to ground 226.

After the output of RF oscillator 140 (FIG. 2) has stabilized in frequency and amplitude, the hold signal at hold input 246 is de-asserted, which causes hold switch 340 to connect the output 234 of RF detector 230 to capacitor 342. Additionally, the reset signal at reset input 248 is de-asserted. This causes reset switch 344 to disconnect output 244 from ground 226 and causes the initialization switch 374 to disconnect node 378 from ground 226. Current from RF detector 230 progressively charges capacitor 342 and the local output signal at output 244 increases. Since capacitor 372 is in a discharged state, the voltage at node 378 follows the increasing local output signal on output 244. Once the voltage on the gate of transistor 376 exceeds the threshold voltage of the transistor, the transistor begins to conduct, which pulls the voltage on node 378 towards ground 226. As capacitor 342 partially discharges into capacitor 372, the local output signal on output 244 falls to a level below the level corresponding to the peak amplitude at the RF output signal V_{F} output by RF filter 220 at output 224. Once transistor 376 conducts, current from RF detector 230 charges capacitors 342, 372 to a voltage corresponding to the stable amplitude of RF output signal V_{F}. The voltage on capacitors 342, 372 provides local output voltage V_{H.}

After the defined integration time, the hold signal at hold input 246 is re-asserted, which causes hold switch 340 to disconnect capacitors 342 and 372 from the output 234 of RF detector 230. Capacitors 342 and 372 retain the voltage thereon until such time as they are discharged by the assertion of the reset signal at reset input 248 causing reset switch 344 and initialization switch 374 to close. During the time that capacitors 342 and 372 retain the voltage thereon, the local output signal V_{H} at the local output node 214 of mass sensor cell 350 is selected by analog signal selector 150 and is converted to a numerical value by ADC 160, as described above with reference to FIG. 2.

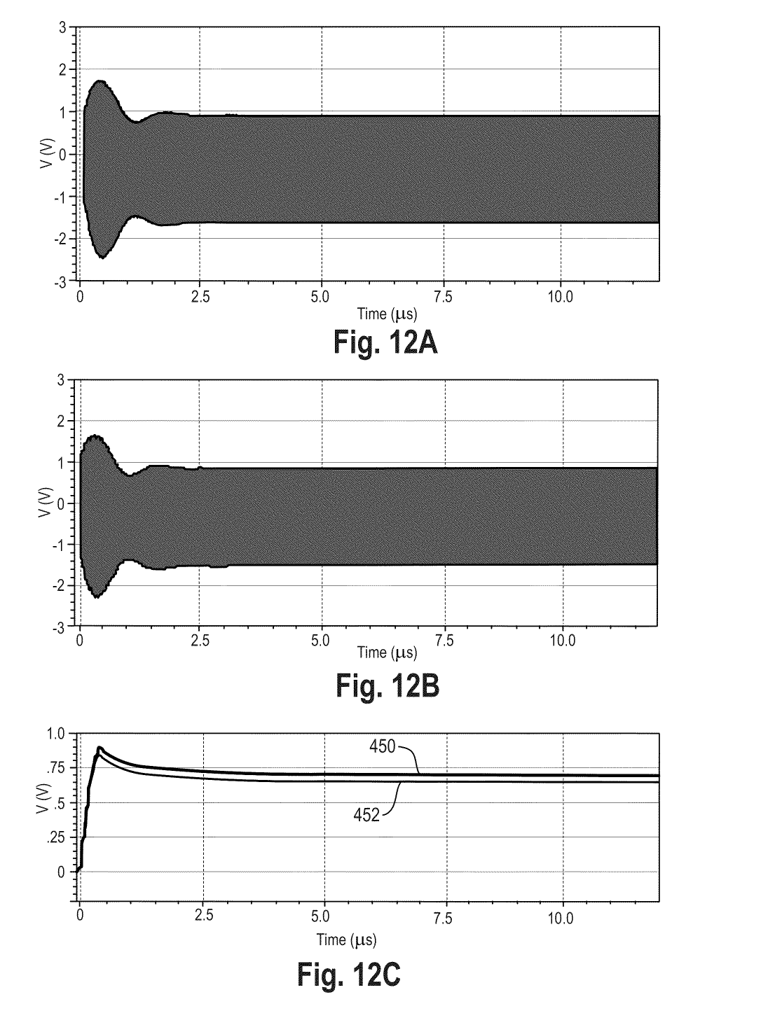

FIGS. 12A-12C are graphs illustrating the time domain response of an example of mass sensor cell 350 incorporating hold circuit 370 to a sine wave input signal over a time span of 15 μs. This is a substantially longer time span than that shown in FIGS. 8A-8C and in FIGS. 10A-100C. In the example shown, hold switch 340 is closed and reset switch 344 and initialization switch 374 are both open. The envelope of a 997.5 MHz input signal with an amplitude of 2V peak to peak applied to input 222 of mass sensor cell 350 is similar to that shown in FIG. 8A. FIG. 12A shows the envelope of RF output signal V_{F} output by mass-dependent RF filter 220 at output 224 prior to an increase in the motional inductance of FBARs 310, 320 (FIG. 3A). The asymmetry exhibited by the waveform is due to the loading imposed on the output of RF filter 220 by capacitors 342, 372 in the positive half cycles during which diode 330 conducts. Once capacitors 342, 372 are charged, the amplitude of the positive half cycles of the output of RF filter 220 stabilizes at about 0.8V. FIG. 12C shows the local output signal V_{H} at the local output node 214 of mass sensor cell 350. In FIG. 12C, trace 450 shows the local output signal V_{H} at local output node 214 prior to the increase in the motional inductance of FBARs 310, 320, local output signal V_{H} stabilizes at about 720 mV approximately 5 μs after the RF input signal is applied to input 222.

FIG. 12B shows the envelope of the RF output signal V_{F} at the output 224 of mass-dependent RF filter 220 after a 0.01% increase in the motional inductance of FBARs 310, 320 (FIG. 3A). This increase in motinal inductance is 1/10 of the increase in motional inductance referred to above in the description of FIGS. 10A-100C. The asymmetry in the waveform is again due to the loading imposed on RF filter 220 by capacitors 342, 372 as diode 330 conducts during positive half cycles of RF output signal V_{F}. In FIG. 12C, trace 452 shows the local output signal V_{H} at local output node 214 after the increase in the motional inductance of FBARs 310, 320, local output signal V_{H} stabilizes at about 640 mV approximately 5 μs after the RF input signal is applied to input 222. In this example, the 0.01% increase in the motional inductance of FBARs 310, 320 (which simulates a much smaller increase in the mass loading of the FBAR than the changes in motional inductance described above with reference to FIGS. 8A-8C and 10A-10C) produces a measurable change in the local output signal V_{H} output by mass sensor cell 350.

In some embodiments, an amplifier (not shown) is interposed between the common analog output 154 of analog signal selector 150 and the analog input 162 of ADC 160. The amplifier is used to subtract from each local output signal output by analog signal selector 150 a voltage equal to the average of the local output signals V_{H} output by mass sensor cells 200 prior to contacting mass sensor 100 with the analyte. The gain of the amplifier is selected to match the input dynamic range of ADC 160 to the anticipated range of the changes in local output signal V_{H} output by mass sensor cells 200 due to mass loading of their constituent FBARs. In applications for determining the presence of a greater than threshold concentration of an analyte, the offset and gain of the amplifier can be configured such that a comparator or a one-bit ADC can be used as ADC 160.

In other embodiments, the level of the RF signal at common RF input 110 is set such that, prior to contacting mass sensor 100 with the analyte, the level of local output signals V_{H} is at or near the full-scale input of ADC 160. Contacting mass sensor 100 with the analyte will only reduce the level of local output signals V_{H}. Moreover, the lowest anticipated level of local output signals V_{H} can be scaled to the minimum input voltage of ADC 160 to maximize effective use of the dynamic range of the ADC. Alternatively, resolution can be improved by increasing gain provided that the noise level remains below the resolution of the ADC.

This disclosure describes the invention in detail using illustrative embodiments. However, the invention defined by the appended claims is not limited to the precise embodiments described.

I claim:

1. A mass sensor, comprising:
a common RF input;
a set of mass sensor cells, each of the mass sensor cells comprising:
a local input node coupled to the common RF input;
a local output node;
an RF filter comprising an electroacoustic resonator having receptors immobilized on a surface thereof,
an RF detector, and
a hold circuit comprising a local hold input,
in which the RF filter, the RF detector and the hold circuit are connected in series between the local input node and the local output node;
a common hold input, the common hold input coupled to the local hold inputs of the hold circuits of at least a subset of the mass sensor cells.

2. The mass sensor of claim 1, in which the electroacoustic resonator comprises a bulk acoustic wave (BAW) device.

3. The mass sensor of claim 2, in which the BAW device is a transverse-mode BAW device.

4. The mass sensor of claim 2, in which the BAW device is a longitudinal-mode BAW device.

5. The mass sensor of claim 2, in which the BAW device comprises an FBAR.

6. The mass sensor of claim 2, in which the BAW device comprises a solidly-mounted resonator.

7. The mass sensor of claim 1, in which the receptors are configured to bond to a target analyte in a fluid sample.

8. The mass sensor of claim 1, in which the electroacoustic resonator is series connected.

9. The mass sensor of claim 1 in which the electroacoustic resonator is shunt connected.

10. The mass sensor of claim 1, in which the RF detector comprises an amplitude detector.

11. The mass sensor of claim 1, in which the RF detector comprises a phase detector.

12. The mass sensor of claim 1, in which the RF detector has an integrating characteristic.

13. The mass sensor of claim 1, in which the hold circuit has an integrating characteristic.

14. The mass sensor of claim 1, additionally comprising an analog signal selector, comprising:
a respective analog signal input connected to the local output node of each of the sensor cells;
a common analog output; and
an address input to receive an address signal defining a specific one of at least a subset of the mass sensor cells whose local output node the analog signal selector is to connect to the common analog output.

15. The mass sensor of claim 14, additionally comprising an analog-to-digital converter comprising an input connected to the common analog output of the analog signal selector.

16. The mass sensor of claim 1, additionally comprising an RF oscillator having an output coupled to the common RF input.

17. The mass sensor of claim 1, additionally comprising an RF amplifier interposed between the common RF input and the local input nodes of at least a subset of the mass sensor cells.

18. The mass sensor of claim 1, in which the mass-dependent RF filter comprises:
an input;
an output;
a series film bulk acoustic resonator (FBAR) connected between the input and the output; and
a shunt FBAR connected between the output and ground.

19. A system for assaying a target analyte in a fluid sample, the system comprising:
an RF oscillator,
a mass sensor, comprising:
a common RF input coupled to the RF oscillator,
a set of mass sensor cells, each of the mass sensor cells comprising:
a local input node connected to the common RF input;
a local output node;
an RF filter comprising an electroacoustic resonator having receptors configured for bonding to the target analyte immobilized on a surface thereof;
an RF detector; and
a hold circuit comprising a local hold input; the RF filter, the RF detector and the hold circuit connected in series between the local input node and the local output node,
a common hold input coupled to the local hold inputs of the hold circuits of a least a subset of the mass sensor cells;
an analog-to-digital converter; and
an analog signal selector, comprising:
a respective analog input connected to the output node of each of at least a subset of the mass sensor cells,
a common analog output coupled to the analog-to-digital converter, and
an address input to receive an address signal defining a specific one of the mass sensor cells whose output node the analog signal selector is to connect to the analog-to-digital converter.

20. The system of claim 19, in which the electroacoustic resonator comprises an FBAR.

21. The system of claim 20, in which the FBAR is a transverse-mode FBAR.

22. The mass sensor of claim 19, in which the electroacoustic resonator is shunt connected.

23. The mass sensor of claim 19, in which the mass-dependent RF filter comprises:
an input;
an output;
a series film bulk acoustic resonator (FBAR) connected between the input and the output; and
a shunt FBAR connected between the output and ground, the shunt FBAR differing in mass from the series FBAR.

24. The system of claim 23, in which:
each of the FBARs has a respective series resonant frequency and a respective parallel resonant frequency, the series resonant frequency and the parallel resonant frequency of the series FBAR differing from the series resonant frequency and the parallel resonant frequency, respectively, of the shunt FBAR; and
the oscillator is to output a frequency intermediate between the parallel resonant frequency of the series FBAR and the parallel resonant frequency of the shunt FBAR.
